(19) United States
(12) Patent Application Publication
(10) Pub. No.: US 2011/0251568 A1
(43) Pub. Date: Oct. 13, 2011

(54) PUNCTAL PLUGS FOR CONTROLLED RELEASE OF THERAPEUTIC AGENTS

(76) Inventors: Nathan R.F. Beeley, Santa Barbara, CA (US); Bret A. Coldren, Vista, FL (US)

(21) Appl. No.: 13/043,171

(22) Filed: Mar. 8, 2011

Related U.S. Application Data
(60) Provisional application No. 61/322,127, filed on Apr. 8, 2010.

(51) Int. Cl. A61F 9/00

(52) U.S. Cl. 604/294

(57) ABSTRACT

Disclosed are lacrimal inserts and their method of use for delivery of medication to the eye. The plug includes a body portion sized to pass through a lacrimal punctum and be positioned within a lacrimal canaliculus of the eyelid. The plug may contain a core, or reservoir, at least partially within the body portion comprising a therapeutic agent that is configured for controlled release into the eye by means of an osmotic engine.

### CROSS REFERENCE TO RELATED APPLICATIONS

This application relates to U.S. patent application Ser. No. 16/322,127, filed Apr. 8, 2010; all applications are herein incorporated by reference in their entireties.

### FIELD OF THE INVENTION

This invention relates to an ophthalmic insert and method for the release of medication to the eye for the treatment of eye disorders. More specifically, the invention relates to punctal plugs sized to pass through a lacrimal punctum and be positioned within a lacrimal canaliculus of the eyelid and containing medication for controlled release into the eye.

### BACKGROUND OF THE INVENTION

Active agents frequently are administered to the eye for the treatment of ocular diseases and disorders. Conventional means for delivering active agents to the eye involve topical application to the surface of the eye. The eye is uniquely suited to topical administration because, when properly constituted, topically applied active agents can penetrate through the cornea and rise to therapeutic concentration levels inside the eye. Active agents for ocular diseases and disorders may be administered orally or by injection, but such administration routes are disadvantageous in that, in oral administration, the active agent may reach the eye in too low a concentration to have the desired pharmacological effect and their use is complicated by significant, systemic side effects and injections pose the risk of infection.

The majority of ocular active agents are currently delivered topically using eye drops which, though effective for some applications, are inefficient. When a drop of liquid is added to the eye, it overfills the conjunctival sac, the pocket between the eye and the lids, causing a substantial portion of the drop to be lost due to overflow of the lid margin onto the cheek. In addition, a substantial portion of the drop that remains on the ocular surface is drained into the lacrimal puncta, diluting the concentration of the drug.

To compound the problems described above, patients often do not use their eye drops as prescribed. Often, this poor compliance is due to an initial stinging or burning sensation caused by the eye drop. Certainly, instilling eye drops in one's own eye can be difficult, in part because of the normal reflex to protect the eye. Therefore, sometimes one or more drops miss the eye. Older patients may have additional problems instilling drops due to arthritis, unsteadiness, and decreased vision, and pediatric and psychiatric patient populations pose difficulties as well.

It is known to use devices that may be inserted into one or more of an orifice of an individual's eye, such as a lacrimal punctum, to deliver active agents. One disadvantage of using such devices to deliver agents is that much of the agent may delivered in an initial, large bolus upon insertion of the device into the eye rather than a more linear delivery of the agent over time.

Prior topical sustained release systems include gradual release formulations, either in solution or ointment form, which are applied to the eye in the same manner as eye drops but less frequently. Such formulations are disclosed, for example, in U.S. Pat. No. 3,826,258 issued to Abraham and U.S. Pat. No. 4,923,699 issued to Kaufman. Due to their method of application, however, these formulations result in many of the same problems detailed above for conventional eye drops. In the case of ointment preparations, additional problems are encountered such as a blurring effect on vision and the discomfort of the sticky sensation caused by the thick ointment base.

Alternatively, sustained release systems have been configured to be placed into the conjunctival cul-de-sac, between the lower lid and the eye. Such units typically contain a core drug-containing reservoir surrounded by a hydrophobic copolymer membrane which controls the diffusion of the drug. Examples of such devices are disclosed in U.S. Pat. No. 3,618,604 issued to Ness, U.S. Pat. No. 3,626,940 issued to Zaffaroni, U.S. Pat. No. 3,845,770 issued to Theeuwes et al., U.S. Pat. No. 3,962,414 issued to Michaels, U.S. Pat. No. 3,993,071 issued to Higuchi et al., and U.S. Pat. No. 4,014,335 issued to Arnold. However, due to their positioning, the units are uncomfortable and poor patient acceptance is again encountered.

### BRIEF DESCRIPTION OF THE DRAWINGS

FIG. 1 shows a cross-sectional view of a lacrimal device according to an illustrative embodiment of the invention positioned in a lacrimal punctum and extending into the lacrimal canaliculus.

FIG. 1A shows a depiction of a pulsatile drug delivery profile over time, for a single therapeutic agent released from a lacrimal plug.

FIG. 1B shows a depiction of pulsatile drug delivery profile over time, for a drug release from a lacrimal plug according to the present invention that contains two therapeutic agents.

FIG. 2A illustrates, in cross-section, a lacrimal plug with an expandable material placed concentrically around a core comprising a therapeutic agent.

FIG. 2B depicts the lacrimal plug of FIG. 2A wherein the expandable material swells to displace therapeutic agent from the core.

FIG. 3 illustrates a cross-sectional view another embodiment of a lacrimal plug according to the present invention.

FIG. 4 illustrates a cross-sectional view of another embodiment of a lacrimal plug according to the present invention that includes a drug core housing and is configured for insertion into the lacrimal punctum and for extending into the lacrimal canaliculus.

FIG. 5A illustrates in cross-section another embodiment of a lacrimal plug according to the present invention having lacrimal fluid inlet pores and is shown inserted in the lacrimal punctum.

FIG. 5B depicts the lacrimal plug of FIG. 5A after activation of the device by water or lacrimal fluid.

FIG. 6 is an illustration of the lacrimal drainage system of the human eye.

FIG. 6A is an illustration of the lacrimal drainage system of the human eye with lacrimal plugs inserted into each punctum with the palpebral fissure in the open position.

FIG. 6B is an illustration of the upper and lower puncta of the human eye with lacrimal plugs inserted into each punctum with the palpebral fissure in the closed position.

FIG. 7 illustrates a distance dependent interaction field between complementary upper and lower lacrimal plug devices when placed in the human eye with a closed palpebral fissure.

FIG. 8 shows a cross-sectional view of an exemplary lacrimal plug according to an embodiment of the present invention having a switchable valve or membrane.

### DETAILED DESCRIPTION OF ILLUSTRATIVE EMBODIMENTS OF THE INVENTION

Punctal plugs have been in use for decades now to treat conditions of dry eye. More recently they have gained attention for use as drug delivery systems for the treatment of ocular diseases and conditions. Several challenges exist with formulating a drug to release at the desired daily rate and or dose that will give efficacy while limiting adverse events.

Diffusion based drug delivery systems are characterized by release rate of drug is dependent on its diffusion through inert water insoluble membrane barrier. There are basically diffusion designs: Reservoir devices and matrix devices. Reservoir devices are those in which a core of drug is surrounded by polymeric membrane. The nature of membrane determines the rate of release of drug from system. The process of diffusion is generally described by a series of equations governed by Fick's first law of diffusion. A matrix device consists of drug dispersed homogenously throughout a polymer.

Reservoir and matrix drug delivery systems are considered diffusion based sustained release systems and constitute any dosage form that provides medication over an extended period of time. The goal of a sustained release system is to maintain therapeutic levels of drug for an extended period and this is usually accomplished by attempting to obtain zero-order release from the sustained release system. Sustained release systems generally do not attain this type of release profile but try to approximate it by releasing in a slow first order manner. Over time, the drug release rate from reservoir and matrix sustained release systems will decay and become non therapeutic.

Zero-order drug release constitutes drug release from a drug delivery system at a steady sustained drug release rate, that is, the amount of drug that is released from the drug delivery system over equal time intervals does not decay and remains at the therapeutic level. This “steady sustained release drug delivery system” is referred to as a zero-order drug delivery system and has the potential to provide actual therapeutic control by its controlled release.

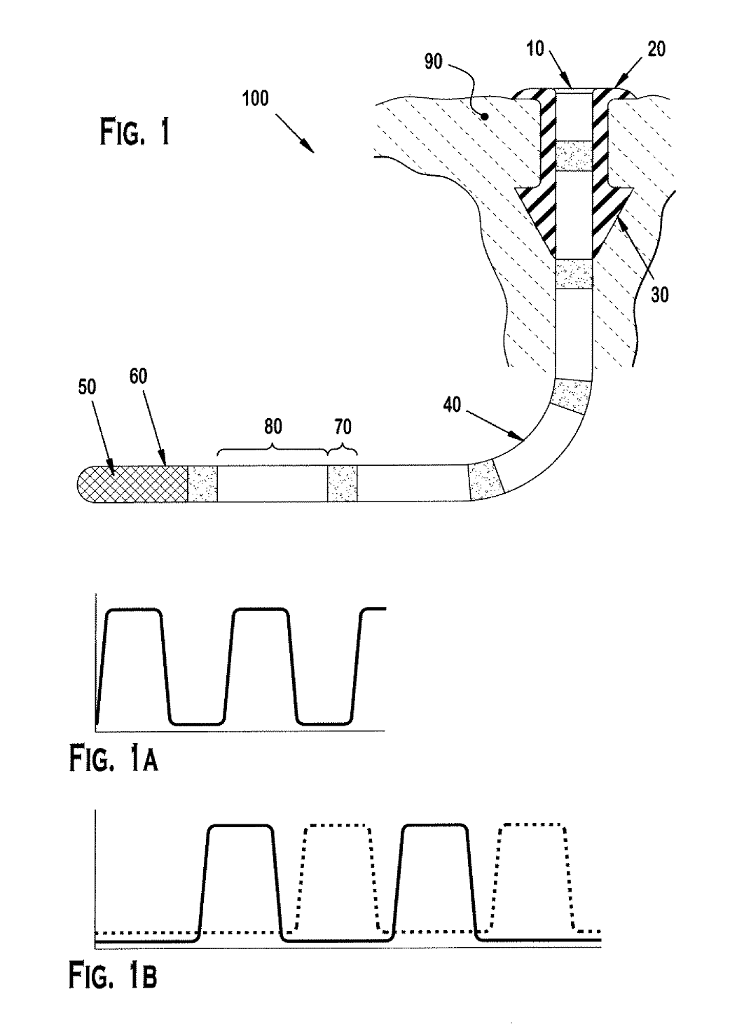

Another drug release profile is referred to as pulsatile drug delivery. Pulsatile drug delivery is intended to release a therapeutic amount of a therapeutic agent at regular intervals. Turning now to the drawing figures, which are meant to be instructive, but not exhaustive of the possible structure and materials of the embodiments of the present invention and wherein similar reference numerals refer to similar structure, an exemplary device illustrative a punctal plug configured for pulsatile release of a therapeutic agent is shown in FIG. 1.

In FIG. 1, one possible embodiment of a punctal plug 100 configured for pulsatile drug delivery is shown. The punctal plug 100 may include a first end 20 having a flange or flange-like cross-sectional profile and a second end 30 having a v-shaped or arrowhead profile. Between the first end 20 and second end 30 a drug-impermeable housing 40 may be provided. When used for drug delivery, an opening in the first end 20 may be equipped with a drug-diffusion limiting member 10. The drug-diffusion limited member 10 may have pores and/or be made of a macroporous membrane structure for permitting a first therapeutic agent formulation 70, a second agent 80 (which may be therapeutic or placebo), or both to pass there through.

FIG. 1 also shows an osmotic engine 50 that be made, at least partially if not wholly, of a water-expandable material that swells when contacted with water or lacrimal fluid. Water or lacrimal fluid may come into contact with the osmotic engine 50 by the inclusion of a semi-permeable water ingress structure 60. As a result of the swelling of the osmotic engine 50, the first therapeutic agent formulation 70, the second agent 80 (which may be therapeutic or placebo), or both may be forced through the drug-impermeable housing 40 and effused via the drug-diffusion limiting member 10. Once effused, the therapeutic agent or agents may then disperse into the lacrimal fluid to provide treatment to the eye.

FIGS. 1A and 1B, by way of illustration, provide exemplary drug release profiles for a pulsatile delivery system. In FIG. 1A, for example, a single therapeutic agent 70 is released in a pulsatile manner, as indicated by the profile that approximates (but need not be identical to) a square-wave. FIG. 1B illustrates pulsatile release of two therapeutic agents. Since these drawing figures are intended to be illustrative, but non-limited, those skilled in the art will be able to envision a pulsatile delivery system that may include one or many active agents and how the resulting release profile may appear. In FIG. 4, a similar device is shown wherein only a single therapeutic agent is contained within the drug-impermeable housing 40.

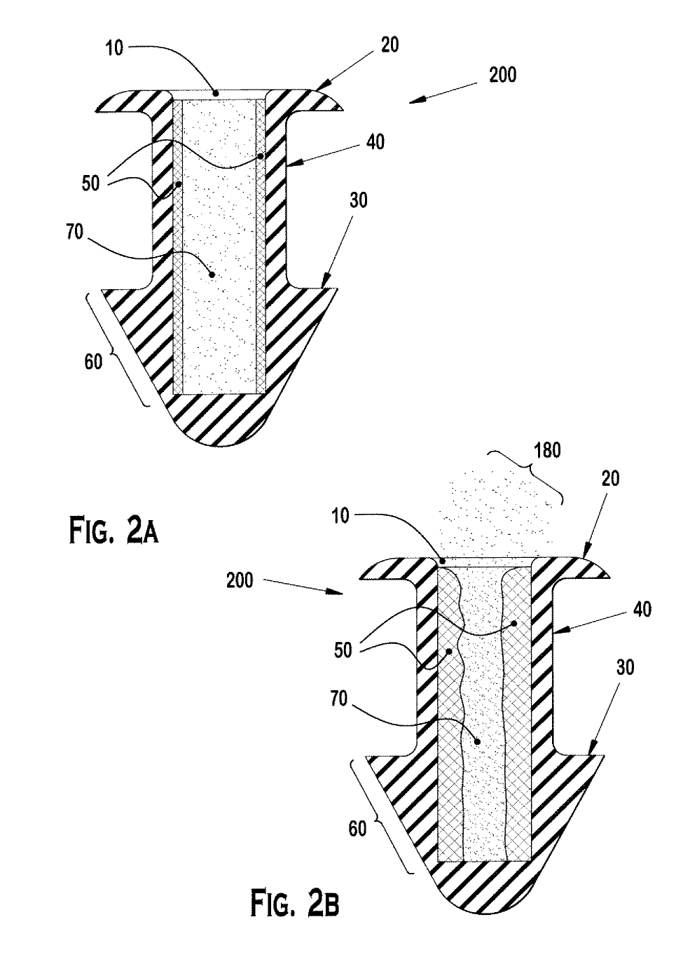

The present invention provides devices that can be used to deliver active agents to the eye in a controlled manner, as shown in FIGS. 2A and 2B. In one embodiment, the invention comprises an ophthalmic device 200 that may include, at least, a body having a first end 20 and a second end 30; a surface extending between the two ends 40 that may comprise drug-impermeable material; and a reservoir 70 contained within the body wherein the reservoir may contains a therapeutic agent for treating a medical condition in the eye.

FIG. 2A illustrates an exemplary device that includes a drug-diffusion limiting member 10 wherein the therapeutic agent in the reservoir 70 may be effused through the drug-diffusion limiting member 10 by action of the osmotic engine 50. As further illustrated in FIG. 2B, when the osmotic engine 50 comes into contact with water or lacrimal fluid, it absorbs the water and swells. The swelling action exerts pressure against the reservoir 70 of therapeutic agent, thus forcing it to be effused via the drug-diffusion limiting member 10.

In one embodiment, the invention provides punctal plugs that may be used to deliver active agents to one or both of the nasolacrimal ducts and to the tear fluid of the eye. An exemplary embodiment may provide a punctal plug having structure, or substantially similar structure, as follows: a body having a first end and a second end; a surface extending between the two ends; and a reservoir contained within the body. An active agent may be present in a continuous or discontinous concentration gradient within an active agent-containing material in the reservoir and eluded by action of an osmotic engine, as illustrated in FIG. 1.

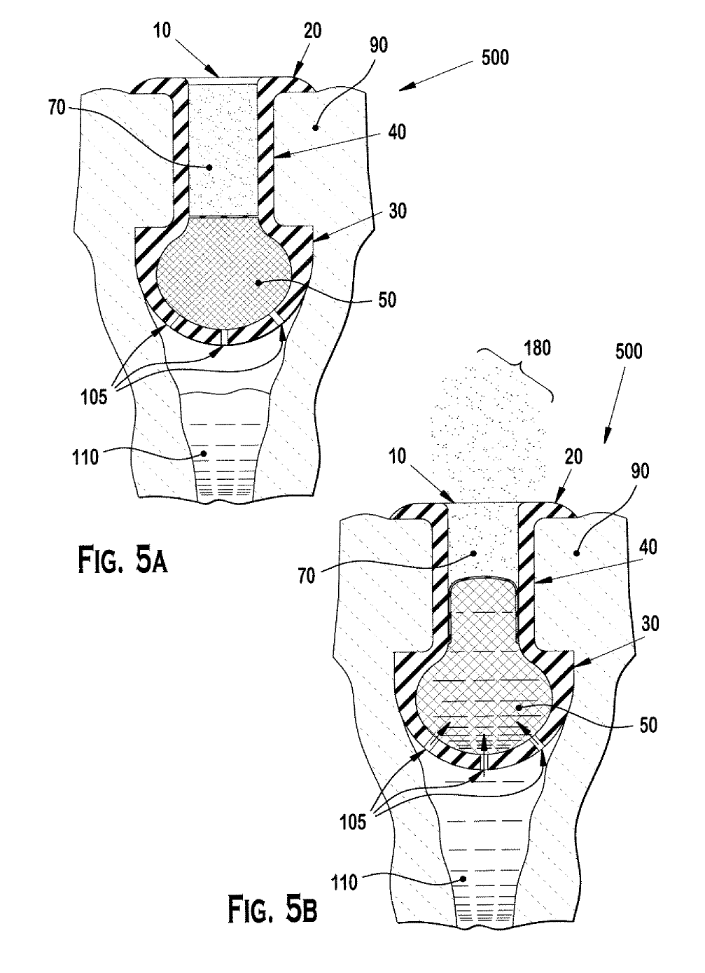

In FIG. 5A, another exemplary embodiment of the invention is illustrated and shows and shows lacrimal fluid inlet pores 105 disposed at points on the retention structure 30 of the punctal plug 500. In this embodiment, lacrimal fluid may enter the housing 40 via the pores 105 to contact the osmotic engine 50. Upon contact with the osmotic engine 50, and as illustrated in FIG. 5B, the osmotic engine swells, thereby causing effusion of the therapeutic agent 180 into the eye via the membrane structure 10.

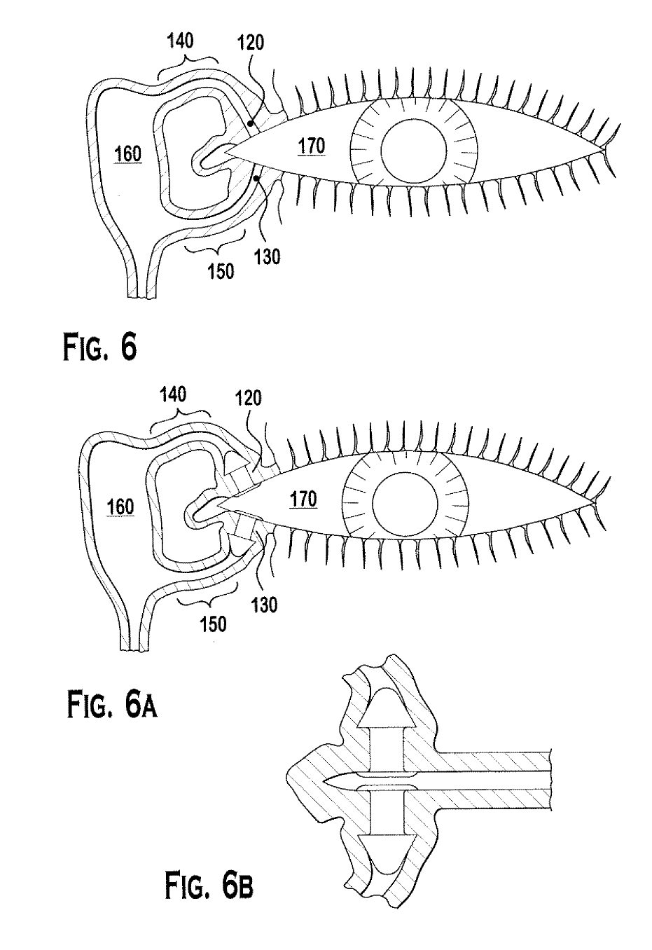

FIGS. 6, 6A, and 6B illustrate exemplary placement of punctal plugs (FIG. 6a) in the upper and lower lacrimal puncta (120, 130) and extending into the upper and lower canaliculus (140, 150). This arrangement places the punctal plugs in fluid communication with lacrimal fluid that may be contained in the lacrimal sac 160.

As used herein, the term “active agent” refers to an agent capable of treating, inhibiting, or preventing a disorder or a disease. Exemplary active agents include, without limitation, pharmaceuticals and nutraceuticals. Preferred active agents are capable of treating, inhibiting, or preventing a disorder or a disease of one or more of the eye, nose and throat.

As used herein, the term “punctal plug” refers to a device of a size and shape suitable for insertion into the inferior or superior lacrimal canaliculus of the eye through, respectively, the inferior or superior lacrimal punctum. Exemplary and illustrative devices are disclosed in U.S. Pat. No. 6,196,993 and U.S. Published Patent Application No. 20090306608A1, both of which are hereby incorporated by reference in their entireties.

As used herein, the term “opening” refers to an opening in the body of a device of the invention of a size and shape through which the active agent can pass. Preferably, only the active agent and formulation can pass through the opening. The opening may be covered with a membrane, single or multiple pores, mesh, grid or it may be uncovered. The membrane, mesh, or grid may be one or more of porous, semi-porous, permeable, semi-permeable, and biodegradable.

The devices of the invention have a reservoir in which is found an active agent-containing material and an active agent therein. The active agent may be dispersed throughout the active agent-containing material or dissolved within the material. Alternatively, the active agent may be contained in inclusions, particulates, droplets, or micro-encapsulated within the material. Still as another alternative, the active agent may be covalently bonded to the material and released by hydrolysis, enzymatic degradation and the like. Yet as another alternative, the active agent may be in a reservoir within the material.

It is a discovery of the invention that the active agent may be released in a controlled manner, meaning over a period of time by using an active agent-containing material in which the agent is present in a substantially continuous concentration gradient throughout the material or by using a discontinuous concentration gradient. This is in contrast to a device that exhibits a therapeutically significant “burst” or immediate release upon insertion of an amount of active agent that is greater than the average release rate over time. According, the mechanical structure described herein may be employed in, but not limited to, a single chamber osmotic pump, elementary osmotic pump, multi chamber osmotic pump, push pull osmotic pump, osmotic pump with non expanding second chamber, controlled porosity osmotic pump, osmotic bursting osmotic pump, delayed delivery osmotic pump, telescopic pump, and/or monolithic osmotic systems.

Without being bound to any particular theory, it is believed that an active agent-containing material that does not undergo significant chemical degradation during the time desired for the release of active agent will release the agent by diffusion through the matrix to a device's release surfaces, meaning surfaces of the active agent-containing material in contact with a person's body fluid. According to Fick's Law, the diffusive transport or flux, J, of the agent through the active agent-containing material is governed at each point and each time by the local concentration gradient, the diffusivity of the active agent with the material D, and the spatial variation of the cross-sectional geometry of the device.

The local gradient may be controlled by placing more active agent at one location in the active agent-containing material relative to another location. For example, the concentration profile can be a continuous gradient from one end of the material to the other. Alternatively, the matrix may be have a discontinuous gradient, meaning that one section of the material has a first concentration and the concentration abruptly changes to a second, different concentration in an adjacent section of the matrix, such as that illustrated in alternative embodiments in FIGS. 1 and 4 as being contained in the drug impermeable housing 40. The diffusivity for the active agent may also be spatially controlled by varying one or more of the chemical composition, porosity, and crystallinity of the active agent-containing material.

Additionally, the spatial variation of the material's cross-sectional geometry may be used to control diffusivity. For example, if the material was in the form of a straight rod that has a uniform active agent concentration, diffusivity will be reduced when the area at the open end of the material is significantly smaller than the average of the entire material. Preferably, the material area at the open end of the device is no more than one-half of the average cross sectional area of the material, meaning the cross section determined perpendicular to the primary dimension of active agent transport use. For illustration, FIG. 7 shows a possible arrangement of the distance dependent interaction field around a punctal plug inserted into a lacrimal punctum.

One of ordinary skill in the art will recognize that, depending on how one varies one or more of the local concentration gradient, the diffusivity of the active agent from the material, and the spatial variation of the cross-sectional geometry of the device, a variety of release profiles may be obtained including, without limitation first order, second order, biphasic, pulsatile and the like. For example, either or both of the active agent concentration and diffusivity may increase from the surface to the center of the active agent-containing material in order to achieve more initial release. Alternatively, either or both may be increased or decreased and then increased again within the material to achieve a pulsatile release profile. The ability to achieve a variety of release profiles by varying local concentration gradient, the diffusivity of the active agent, and the spatial variation of the cross-sectional geometry may eliminate the need for rate-limiting membranes in the device. The device may further comprise a switchable valve (also referred to herein as a modulating element) 190 in the opening in the housing 40 to give the designer greater control over the drug elusion profile of the device.

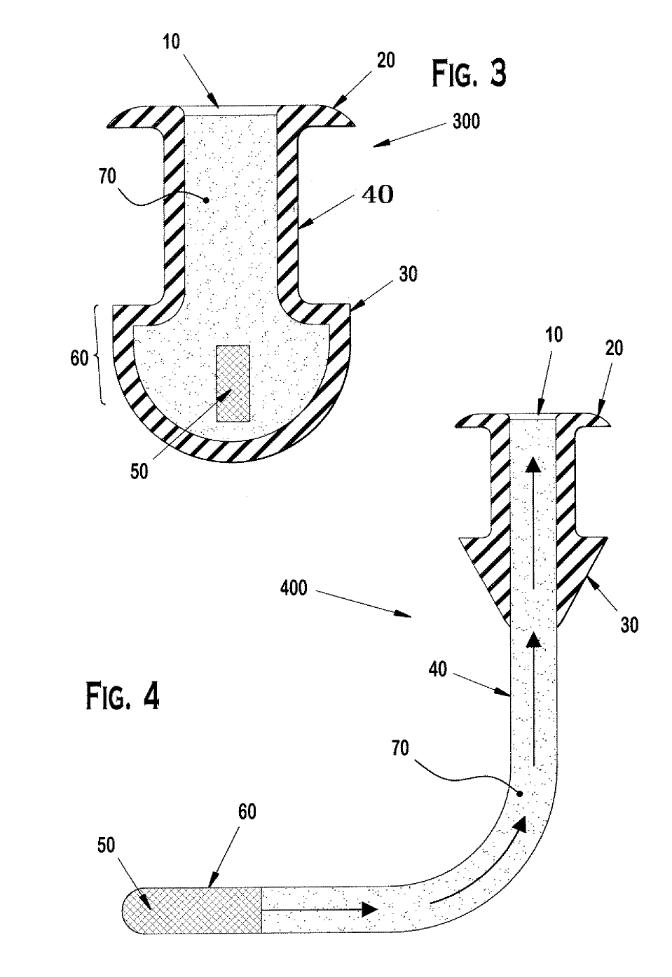

The devices of the invention contain a reservoir within the body, and the reservoir contains at least one active agent-containing material, as shown in an exemplary embodiment in FIG. 3. The body 40 is preferably impermeable to the active agent, meaning only an insubstantial amount of active agent can pass there through, and the body has at least one opening 10 through which the active agent is released. The active agent-containing material 70 useful in the devices of the invention is any material that is capable of containing the active agent, does not alter the chemical characteristics of the active agent, and does not significantly chemically degrade or physically dissolve when placed in contact with ocular fluids. Preferably, the active agent-containing material is non-biodegradable, meaning that it does not degrade to a substantial degree upon exposure to biologically active substances typically present in mammals. Additionally, the active agent-containing material is capable of releasing the active agent by one or more of diffusion, degradation, or hydrolyzation. Preferably, the active agent-containing material is a polymeric material, meaning that it is a material made of one or more types of polymers.

When the active agent-containing material is combined with the active agent, thereby forming the material included in the reservoir 70, the material may also contain one or more materials that are insoluble in water and non-biodegradable, but from which the active agent can diffuse. For example, if the active agent-containing material is a polymeric material, the material may be composed of one or more polymers that are insoluble in water and non-biodegradable.

The mechanism by which the therapeutic agent is effused is illustrated in one exemplary embodiment in the device of FIG. 3. FIG. 3 shows the osmotic engine 50 that swells upon contact with water or lacrimal fluid. The swelling of the osmotic engine 50 cause effusion of the therapeutic agent from the reservoir 70 through the drug-diffusion limiting membrane 10.

Suitable polymeric materials for the active agent-containing material include, without limitation, hydrophobic and hydrophilic absorbable and non-absorbable polymers. Generally, liquid, gel and other soluble drug formulations are preferred. Alternatively, suitable hydrophobic, non-absorbable polymers include, without limitation, ethylene vinyl alcohol (“EVA”), fluorinated polymers including without limitation, polytetrafluoroethylene (“PTFE”) and polyvinylidene fluoride (“PVDF”), polypropylene, polyethylene, polyisobutylene, nylon, polyurethanes, polyacrylates and methacrylates, polyvinyl palmitate, polyvinyl stearates, polyvinyl myristate, cyanoacrylates, epoxies, silicones, copolymers thereof with hydrophobic or hydrophilic monomers, and blends thereof with hydrophilic or hydrophobic polymers and excipients.

Hydrophilic, non-absorbable polymers useful in the invention include, without limitation, cross-linked poly(ethylene glycol), poly(ethylene oxide), polypropylene glycol), poly(vinyl alcohol), poly(hydroxyethyl acrylate or methacrylate), poly(vinylpyrrolidone), polyacrylic acid, poly(ethyloxazoline), and poly(dimethyl acrylamide), copolymers thereof with hydrophobic or hydrophilic monomers, and blends thereof with hydrophilic or hydrophobic polymers and excipients.

Hydrophobic, absorbable polymers that may be used include, without limitation, aliphatic polyesters, polyesters derived from fatty acids, poly(amino acids), poly(ether-esters), poly(ester amides), polyalkylene oxalates, polyamides, poly(iminocarbonates), polycarbonates, polyorthoesteres, polyoxaesters, polyamidoesters, polyoxaesters containing amine groups, phosphoesters, poly)anhydrides), polypropylene fumarates, polyphosphazenes, and blends thereof. Examples of useful hydrophilic, absorbable polymers include, without limitation, polysaccharides and carbohydrates including, without limitation, crosslinked alginate, hyaluronic acid, dextran, pectin, hydroxyethyl cellulose, hydroxy propyl cellulose, gellan gum, guar gum, keratin sulfate, chondroitin sulfate, dermatan sulfate, proteins including, without limitation, collagen, gelatin, fibrin, albumin and ovalbumin, and phospholipids including, without limitation, phosphoryl choline derivatives and polysulfobetains.

In one possible embodiment, the active agent-containing material is a polymeric material that is polycaprolactone. In still another, the material is poly(epsilon-caprolactone), and ethylene vinyl acetate of molecular weights between about 10,000 and 80,0000. About 0 to about 100 weight percent polycaprolactone and about 100 to about 0 weight percent of the ethylene vinyl acetate are used based on the total weight of the polymeric material and, as well, about 50% each of polycaprolactone and ethylene vinyl acetate is used.

The polymeric material used may be greater than about 99% pure and the active agents may be greater than about 97% pure. One of ordinary skill in the art will recognize that in compounding, the conditions under which compounding is carried out will need to take into account the characteristics of the active agent to ensure that the active agents do not become degraded by the process. The polycaprolactone and ethylene vinyl acetate preferably are combined with the desired active agent or agents, micro-compounded, and then extruded.

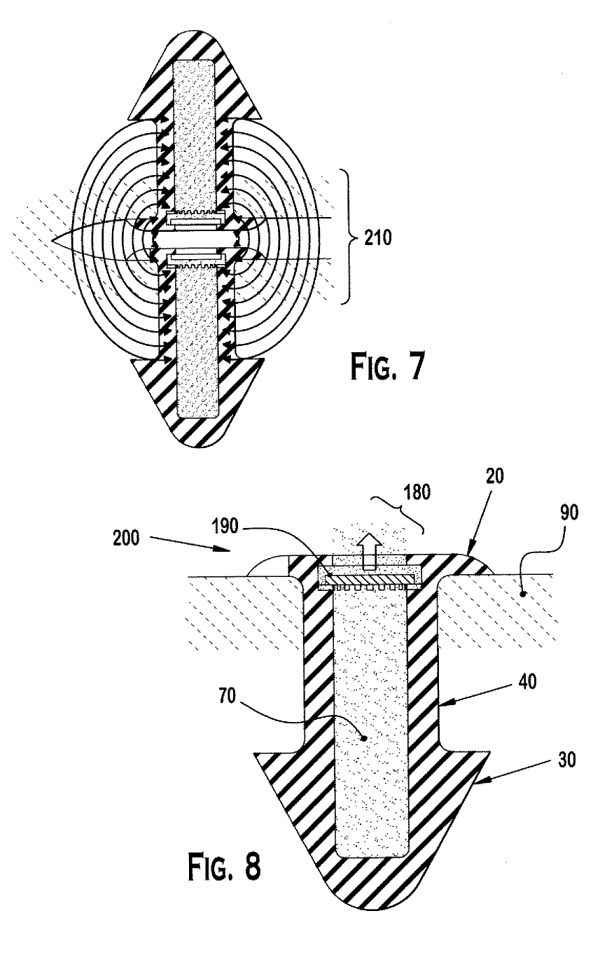

In the devices of the invention, a release-modulating component may be included. The release-modulating component may be any component that acts to modulate the release of the active agent from the plug. Suitable modulating component include, without limitation, one or more biodegradable of non-biodegradable semi-permeable membrane, one or more pores, or combinations thereof. In FIG. 8 is shown an embodiment of the invention in which there is a modulating component 190. As depicted, punctal plug 200 has reservoir 70 with an opening in the support flange 20. In addition to a gradient, release of the active agent may be controlled by use of one or both of active agent loading and release enhancers or, as shown in FIGS. 1, 2A, 2B, 3, 4, 5A, and 5B, an osmotic engine 50.

In addition to or instead of active agent loading profiles, the release kinetics may be controlled via spatial gradients of the properties of degradability and drug permeability of the active agent-containing material. For example, in those cases in which drug release kinetics are dominated by the rate of material degradation, a spatial degradation in the material chemistry including, without limitation, polylactide-glycolide copolymers of differing monomer ratios, adjacent polyglycolide and polycaprolactone layers and the like, results in spatial gradients and varied release rates as the material degradation front moves through the device. By way of further example, a material may erode more slowly initially in a first, outer material and more quickly in a second, inner material to achieve phased release kinetics.

In the case of a non-degradable material that elutes the active agent solely through diffusion-dominated mechanisms, spatial gradients in the material's permeability can control release kinetics beyond what is possible with a homogeneous material. In the diffusion-dominated mechanism, the material permeability controls release kinetics and is influenced by the material's porosity as well as the active agent solubility and diffusivity. By forming an active agent-loaded layer of an outer material with a higher permeability, the active agent elution may be controlled to be more linear with less burst effect than that which is otherwise achieved with a single, homogeneous, diffusion material.

The spatial gradients in biodegradability or permeability may be combined with continuous or step-wise gradients in the active agent loading profile. For example, a punctal plug material core having an outer segment loaded with a low active agent concentration and with a relatively low active agent permeability may be adjacent to an inner material segment loaded with a high agent concentration and with a relatively high active agent permeability, which combination achieves release kinetics unobtainable with a homogeneous material ad homogeneous active agent loading. The initial burst release is reduced and the release of the last active agent content is accelerated relative to a conventional homogeneous active agent loaded device.

Phase-separated inclusions may be used to control one or both of diffusive and degradative kinetics of the active agent-containing material. For example, water soluble polymers, water soluble salts, materials with a high diffusivity for the active agent and the like may be used as destabilizing inclusion to enhance degradation or diffusion rates. When the hydrolysis front reaches an inclusion, the inclusion rapidly dissolves and increases porosity of the active agent-containing material. The inclusions may be incorporated as gradients or layers that allow additional tailoring of the release profile.

As another alternative, a percolated network of destabilizing inclusions may be used. When used in a non-biodegradable active agent-containing material, these inclusions form islands within the material that can possess high diffusivity for the active agent. Useful inclusions will have a higher diffusivity for the active agent than the active agent-containing material. Examples of such inclusions include, without limitation, propylene glycol, silicone oil, immiscible dispersed solids such as a polymer or wax and the like. As yet another example, an inclusion that acts to absorb water, swell the active agent-containing material and increase local diffusion kinetics may be used.

As still another alternative, stabilizing inclusions that have a low active agent diffusivity are used. These inclusions act to form a barrier that slows diffusive transport of the active agent in the vicinity of the inclusion. The overall effect is a reduction of active agent permeability in a base material that is otherwise the same. Example of such inclusions include, without limitation, micro to nano-sized silicate particles dispersed through the base material of one or both of polycaprolactone and ethylenecovinylacetate homogeneously or in continuous step-wise gradients.

The present invention encompasses numerous devices for the delivery of active agents to the eye each having various features and advantages. For example, certain devices may have a body with a first end, a second end, and a lateral surface extending between the two ends. The lateral surface preferably has an outer diameter that is substantially circular in shape and, thus, the body preferably has a cylindrical shape. A portion of the lateral surface of certain of the devices preferably has an outer diameter that is greater than the outer diameter of the remainder of the lateral surface as shown in FIG. 1. The enlarged portion can be any size or shape, and can be present on any part of the lateral surface, in punctal plug embodiments, the enlarged portion is of a size so that it at least partially anchors the punctal plug in the lacrimal canaliculus and preferably, the enlarged portion is at one end of the plug. One ordinarily skilled in the art will recognize that any of a wide variety of shapes are possible.

The body of the punctal plugs of the invention may take any shape and size, preferably, the body is in the shape of an elongated cylinder. The body will be about 0.8 to about 5 mm in length, preferably about 1.2 to about 2.5 mm in length. The width of the body will be about 0.2 to about 3, preferably 0.3 to about 1.5 mm. The size of the opening will be from about 1 nm to about 2.5 mm and preferably about 0.15 mm to about 0.8 mm. Instead of one large opening at any one location, multiple small openings may be used. The body of the plug may be wholly or partially transparent or opaque. Optionally, the body may include a tint or pigment that makes the plug easier to see when it is placed in a punctum.

The body of the devices of the invention may be made of any suitable biocompatible material including, without limitation, silicone, silicone blends, silicone co-polymers, such as, for example, hydrophilic monomers of polyhydroxyethylmethacrylate (“pHEMA”), polyethylene glycol, polyvinylpyrrolidone, and glycerol, and silicone hydrogel polymers such as, for example, those described in U.S. Pat. Nos. 5,962,548, 6,020,445, 6,099,852, 6,367,929, and 6,822,016, incorporated herein in their entireties by reference. Other suitable biocompatible materials include, for example: polyurethane; polymethylmethacrylate; poly(ethylene glycol); poly(ethylene oxide); polypropylene glycol); poly(vinyl alcohol); poly(hydroxyethyl methacrylate); poly(vinylpyrrolidone) (“PVP”); polyacrylic acid; poly(ethyloxazoline); poly(dimethyl acrylamide); phospholipids, such as, for example, phosphoryl choline derivatives; polysulfobetains; acrylic esters, polysaccharides and carbohydrates, such as, for example, hyaluronic acid, dextran, hydroxyethyl cellulose, hydroxylpropyl cellulose, gellan gum, guar gum, heparan sulfate, chondroitin sulfate, heparin, and alginate; proteins such as, for example, gelatin, collagen, albumin, and ovalbumin; polyamino acids; fluorinated polymers, such as, for example, PTFE, PVDF, and teflon; polypropylene; polyethylene; nylon; and EVA.

The surface of the devices may be wholly or partially coated. The coating may provide one or more of lubriciousness to aid insertion, muco-adhesiveness to improve tissue compatibility, and texture to aid in anchoring the device. Examples of suitable coatings include, without limitation, gelatin, collagen, hydroxyethyl methacrylate, PVP, PEG, heparin, chondroitin sulphate, hyaluronic acid, synthetic and natural proteins, and polysaccharides, thiomers, thiolated derivatives of polyacrylic acid and chitosan, polyacrylic acid, carboxymethyl cellulose and the like and combinations thereof.

Certain embodiments of the devices of the invention have a body made of a flexible material that conforms to the shape of whatever it contacts. Optionally, in the punctal plug embodiment, there may be a collarette formed of either a less flexible material than that of the body or material that too conforms to the shape of whatever it contacts. When a punctal plug having both a flexible body and a less flexible collarette is inserted into the lacrimal canaliculus, the collarette rests on the exterior of the lacrimal punctum and the body of the punctal plug conforms to the shape of the lacrimal canaliculus. The reservoir and the body of such punctal plugs are preferably coterminous. That is, the reservoir of such punctal plugs preferably make up the entirety of the body, except for the collarette.

In embodiments in which one or both of a flexible body and collarette are used, the flexible body and flexible collarette can be made of materials that include, without limitation, nylon, polyethylene terephthalate (“PET”), polybutylene terephthalate (“PBT”), polyethylene, polyurethane, silicone, PTFE, PVDF, and polyolefins. Punctal plugs made of nylon, PET, PBT, polyethylene, PVDF, or polyolefins are typically manufactured for example and without limitation, extrusion, injection molding, or thermoforming. Punctal plugs made of latex, polyurethane, silicone, or PTFE are typically manufactured using solution-casting processes.

Processes for manufacturing the punctal plugs useful in the invention are well known. Typically, the devices are manufactured by injection molding, cast molding, transfer molding or the like. Preferably, the reservoir is filled with one or both of at least one active agent and the active agent-containing material subsequent to the manufacture of the device. Additionally, one or more excipients may be combined with the active agent alone or in combination with the polymeric material.

The amount of active agent used in the devices of the invention will depend upon the active agent or agents selected, the desired doses to be delivered via the device, the desired release rate, and the melting points of the active agent and active agent-containing material. Preferably, the amount used is a therapeutically effective amount meaning an amount effective to achieve the desired treatment, inhibitory, or prevention effect. Typically, amounts of about 0.05 to about 8,000 micrograms of active agents may be used.

In certain aspects of the invention, the reservoir can be refilled with a material after substantially all of the active agent-containing material has dissolved or degraded and the active agent is released. For example, the new active agent-containing material can be the same as, or different from, the previous polymeric material, and can contain at least one active agent that is the same as, or different from the previous active agent. Certain punctal plugs used for particular applications can preferably be refilled with a material while the punctal plugs remain inserted in the lacrimal canaliculus, while other punctal plugs are typically removed from the lacrimal canaliculus, a new material is added, and the punctal plugs are then reinserted into the lacrimal canaliculus.

After the device is filled with the active agent, the plug is sterilized by any convenient method including, without limitation, ethylene oxide, autoclaving, irradiation, and the like and combination thereof. Preferably, sterilization is carried out through gamma radiation or use of ethylene oxide.

The devices described herein can be used to deliver various active agents for the one or more of the treatment, inhibition, and prevention of numerous diseases and disorders. Each device may be used to deliver at least one active agent and can be used to deliver different types of active agents. For example, the devices can be used to deliver azelastine HCl, emadastine difumerate, epinastine HCl, ketotifen fumerate, levocabastine HCl, olopatadine HCl, pheniramine maleate, and antazoline phosphate for one or more of the treatment, inhibition, and prevention of allergies. The devices can be used to deliver mast cell stabilizers, such as, for example, cromolyn sodium, lodoxamide tromethamine, nedocromil sodium, and permirolast potassium.

The devices can be used to deliver mydriatics and cycloplegics including, without limitation, atropine sulfate, homatropine, scopolamine HBr, cyclopentolate HCl, tropicamide, and phenylephrine HCl. The devices can be used to deliver ophthalmic dyes including, without limitation, rose bengal, sissamine green, indocyanine green, fluorexon, and fluorescein.

The devices can be used to deliver corticosteroids including, without limitation, dexamethasone sodium phosphate, dexamethasone, fluoromethalone, fluoromethalone acetate, loteprednol etabonate, prednisolone acetate, prednisolone sodium phosphate, medrysone, rimexolone, and fluocinolone acetonide. The devices can be used to deliver non-steroidal anti-inflammatory agents including, without limitation, flurbiprofen sodium, suprofen, diclofenac sodium, ketorolac tromethamine, cyclosporine, rapamycin methotrexate, azathioprine, and bromocriptine.

The devices can be used to deliver anti-infective agents including, without limitation, tobramycin, moxifloxacin, ofloxacin, gatifloxacin, ciprofloxacin, gentamicin, sulfisoxazolone diolamine, sodium sulfacetamide, vancomycin, polymyxin B, amikacin, norfloxacin, levofloxacin, sulfisoxazole diolamine, sodium sulfacetamide tetracycline, doxycycline, dicloxacillin, cephalexin, amoxicillin/clavulante, ceftriaxone, cefixime, erythromycin, ofloxacin, azithromycin, gentamycin, sulfadiazine, and pyrimethamine.

The devices can be used to deliver agents for one or more of the treatment, inhibition, and prevention of glaucoma including, without limitation, epinephrines, including, for example: dipivefrin; alpha-2 adrenergic receptors, including, for example, aproclonidine and brimonidine; betablockers including, without limitation, betaxolol, carteolol, levobunolol, metipranolol, and timolol; direct miotics, including, for example, carbachol and pilocarpine; cholinesterase inhibitors, including, without limitation, physostigmine and echothiophate; carbonic anhydrase inhibitors, including, for example, acetazolamide, brinzolamide, dorzolamide, and methazolamide; prostoglandins and prostamides including, without limitation, latanoprost, bimatoprost, uravoprost, and unoprostone cidofovir.

The devices can be used to deliver antiviral agents, including, without limitation, fomivirsen sodium, foscarnet sodium, ganciclovir sodium, valganciclovir HCl, trifluridine, acyclovir, and famciclovir. The devices can be used to deliver local anesthetics, including, without limitation, tetracaine HCl, proparacaine HCl, proparacaine HCl and fluorescein sodium, benoxinate and fluorescein sodium, and benoxnate and fluorexon disodium. The devices can be used to deliver antifungal agents, including, for example, fluconazole, flucytosine, amphotericin B, itraconazole, and ketocaonazole.

The devices used to deliver analgesics including, without limitation, acetaminophen and codeine, acetaminophen and hydrocodone, acetaminophen, ketorolac, ibuprofen, and tramadol. The devices can be used to deliver vasoconstrictors including, without limitation, ephedrine hydrochloride, naphazoline hydrochloride, phenylephrine hydrochloride, tetrahydrozoline hydrochloride, and oxymetazoline. Finally, the devices can be used to deliver vitamins, antioxidants, and nutraceuticals including, without limitation, vitamins A, D, and E, lutein, taurine, glutathione, zeaxanthin, fatty acids and the like.

The active agents delivered by the devices can be formulated to contain excipients including, without limitation, synthetic and natural polymers, including, for example, polyvinylalcohol, polyethyleneglycol, PAA (polyacrylic acid), hydroxymethyl cellulose, glycerine, hypromelos, polyvinylpyrrolidone, carbopol, propyleneglycol, hydroxypropyl guar, glucam-20, hydroxypropyl cellulose, sorbitol, dextrose, polysorbate, mannitol, dextran, modified polysaccharides and gums, phosolipids, and sulphobetains.

In another embodiment of the invention, the punctal plug drug delivery system may produce a steady and/or sustained drug delivery release rate that is driven by a water penetration mechanism that induces an osmotically controlled mechanical displacement using an inorganic water-soluble osmogent such as magnesium sulphate, sodium chloride, sodium sulphate, potassium chloride or sodium bicarbonate, and combinations and mixtures thereof.

In another embodiment of the invention, the punctal plug drug delivery system may produce a steady and/or sustained drug delivery release rate that is driven by a water penetration mechanism that induces an osmotically controlled mechanical displacement using an organic water-soluble osmogent such as sodium carboxymethyl cellulose, hydroxypropylmethyl cellulose, hydroxyethylmethylcellulose, methylcellulose, polyethylene oxide or polyvinyl pyrollidine, polyacrylic acid copolymers, and salts thereof, and combinations and mixtures thereof. These compositions may be used to manufacture a punctal plug that includes a cohesive, hydrogel engine of particular utility with water inlet pores.

In another embodiment of the invention, the punctal plug drug delivery system may produce a steady and/or sustained drug delivery release rate that is driven by a water penetration mechanism that induces an osmotically controlled mechanical displacement that is driven by the tear film fluid or the nasolacrimal canal moisture or a self contained water source.

In another embodiment of the invention, the punctal plug drug delivery system may produce a steady and/or sustained drug delivery release rate that is driven by a water penetration mechanism that induces an osmotically, swelling of chemically controlled mechanical displacement of a membrane, piston or compartment and causes the displacement of alternate zones of active pharmaceutical ingredients and inactive ingredients out of the punctal plug for a pulsatile release profile.

In another embodiment of the invention, the punctal plug drug delivery system may produce a steady and/or sustained drug delivery release rate that is driven by a water penetration mechanism that induces an osmotically, swelling or chemically controlled mechanical displacement of a membrane, piston or compartment and causes the displacement of alternate zones of one active pharmaceutical ingredients and a second or third or more additional zones of additional active ingredients out of the punctal plug for a pulsatile release profile.

In another embodiment of the invention, the punctal plug drug delivery system may produce a steady and/or sustained drug delivery release rate that is driven by a water penetration mechanism that induces an osmotically, swelling or chemically controlled mechanical displacement of a membrane, piston or compartment and causes the displacement of alternate zones of active pharmaceutical ingredients separated by non permeable inactive ingredients out of the punctal plug for a pulsatile release profile.

In another embodiment of the invention, the punctal plug drug delivery system may produce a steady and/or sustained drug delivery release rate that is driven by a water penetration mechanism that induces an osmotically, swelling or chemically controlled mechanical displacement of a membrane, piston or compartment and causes the displacement of alternate zones of active pharmaceutical ingredients out of the punctal plug for a pulsatile release profile.

In another embodiment of the invention, the punctal plug drug delivery system may produce a steady and/or sustained drug delivery release rate that is driven by a water penetration mechanism that induces an osmotically, swelling or chemically controlled mechanical displacement of a membrane, piston or compartment and causes the displacement of alternate zones of active pharmaceutical ingredients and inactive ingredients out of the punctal plug for a pulsatile release profile where the release profile is modulated to give 1 to 96 hours of active release, preferably 1 to 24 hours of active release and 1 to 96 hour inactive release, preferably 8 to 48 h of inactive release throughout a total treatment course of 1 minute to as many as 0.25 to 5 years.

In another embodiment of the invention, the punctal plug drug delivery system may produce a steady and/or sustained drug delivery release rate that is driven by a water penetration mechanism that induces an osmotically, swelling or chemically controlled mechanical displacement of a membrane, piston or compartment and causes the displacement of 0.0001 nanoliters to 100 ml per hour, preferably 0.01 to 1 nanoliter per hour.

In another embodiment of the invention, the punctal plug drug delivery system may produce a steady and/or sustained drug delivery release rate that is driven by a water penetration mechanism that induces an osmotically, swelling or chemically controlled mechanical displacement of a membrane, piston or compartment and causes the displacement of 0.0000001 micro grams to 500 micrograms per hour, preferably 0.1 to 20 micrograms per hour.

In another embodiment of the invention, the punctal plug drug delivery system may produce a steady and/or sustained drug delivery release rate that is driven by a water penetration mechanism that induces an osmotically, swelling or chemically controlled mechanical displacement of a membrane, piston or compartment and causes the displacement of one or more active pharmaceutical ingredients to treat glaucoma, dry eye, infections, inflammation, pain or other ocular disease or condition.

In another embodiment of the invention, the punctal plug drug delivery system may include two punctal plugs located in the upper and lower punctal canal where one contains one or more active pharmaceutical ingredients and a magnetic mechanical valve and the second punctal plug is the magnetic polar opposite and results in a pulsatile opening and closing of the valve during blinking and or sleeping.

In another embodiment of the invention, the punctal plug drug delivery system may produce a steady and/or sustained drug delivery release rate that is driven by a water penetration mechanism that induces an osmotically, swelling or chemically controlled mechanical displacement of a membrane, piston or compartment and also a valve to modulate the flow and pressure of the system.

In another embodiment of the invention, structures and materials may be used to modulate and/or control the rate of water penetration into the expandable material. Exemplary structure may include, but are not limited to, pores, organic and inorganic semipermeable membranes, etc. The rate of expansion of the osmogen, which relates to the rate at which the osmotic engine eludes therapeutic agent from the reservoir, i.e., the displacement/pumping/release rate of the active agent.

In another alternative embodiment, a punctal plug is provided with an osmotic engine, as shown generally in FIG. 3. Noting the absence of a piston, the osmotic engine 50 may comprise a dense hydrogel formulation having sufficient cohesiveness to exhibit at least some properties of a solid. Water ingress structure 60 may be fabricated to include pores or a water-inlet membrane to permit the infusion of water to the osmotic engine 50. The osmotic engine 50, not shown to scale in FIG. 3, may be sized or configured to maintain separation of the drug region (or reservoir 70 of therapeutic agent) and the osmotic pump region. A particularly viscous drug formation can be eluded by this device via membrane 10, which may comprise a membrane, pores, or other structure that permit the discharge of the therapeutic agent from the reservoir 70.

What is claimed is:

1. A punctal plug drug delivery system, comprising a drug impermeable housing having a quantity of a therapeutic agent in a reservoir, a semi-permeable membrane and/or small pores, in fluid communication with the reservoir, and a mechanical displacement engine in mechanical communication with the reservoir for moving the therapeutic agent out of the punctal plug.

2. The punctal plug of claim 1, wherein the mechanical engine is actuated by osmosis, swelling, or chemical reaction.

3. The punctal plug of claim 1, wherein mechanical displacement engine comprises one or more of a single chamber osmotic pump, elementary osmotic pump, multi chamber osmotic pump, push pull osmotic pump, osmotic pump with non expanding second chamber, controlled porosity osmotic pump, osmotic bursting osmotic pump, delayed delivery osmotic pump, telescopic pump, monolithic osmotic systems.

4. The punctal plug of claim 1 wherein osmosis of water through the semi-permeable membrane into the mechanical displacement engine induces a mechanical displacement from a membrane, piston or compartment located in the base of the punctal plug.

5. The punctal plug of claim 2 wherein penetration of water through the semi-permeable membrane induces an osmotically, swelling or chemically controlled mechanical displacement from a membrane, piston or compartment located at the base of an extended tube that extends into the lacrimal duct and or nasolacrimal canal.

6. The punctal plug of claim 2 wherein penetration of water through the semi-permeable member induces an osmotically, swelling or chemically controlled mechanical displacement from a membrane, piston or compartment located in the shaft of the punctal plug.

7. A punctal plug drug delivery system according to claim 2, wherein the therapeutic agent and the osmotic engine are configured for release according to a substantially pulsatile release profile.

8. A punctal plug drug delivery system according to claim 2, wherein the therapeutic agent and the osmotic engine are configured for release according to a substantially continuous release profile.

9. A punctal plug drug delivery system according to claim 2, wherein the therapeutic agent and the osmotic engine are configured for release according to a substantially gradient release profile.

10. The punctal plug according to any of claim 2, wherein the reservoir contains more than one therapeutic agent.

11. A punctal plug drug delivery system, comprising a drug impermeable housing having a quantity of at least one therapeutic agent in a reservoir, a semi-permeable membrane and/or small pores, in fluid communication with the reservoir, and a mechanically or electrically actuated engine or a microelectromechanical engine in mechanical communication with the reservoir for moving the therapeutic agent out of the punctal plug.

12. The punctal plug of claim 1 wherein the engine is microelectromechanical.

13. A punctal plug drug delivery system according to claim 11, wherein the therapeutic agent and the osmotic engine are configured for release according to a substantially pulsatile release profile.

14. A punctal plug drug delivery system according to claim 11, wherein the therapeutic agent and the osmotic engine are configured for release according to a substantially continuous release profile.

15. A punctal plug drug delivery system according to claim 11, wherein the therapeutic agent and the osmotic engine are configured for release according to a substantially gradient release profile.

16. A punctal plug drug delivery system comprising a drug impermeable housing having a reservoir and a drug delivery engine, wherein the reservoir contains one or more therapeutic agents and the drug delivery engine is selected from one of an osmotic engine and a microelectromechanical engine.

17. The punctal plug of claim 16 configured to release drug substantially according to a pulsatile, continuous, or gradient profile.

18. The punctal plug of claim 16 wherein the one or more therapeutic agents comprise therapeutically effective drugs for the treatment, inhibition, and prevention of glaucoma.

19. The punctal plug of claim 18 wherein the one or more therapeutic agents are selected from one or more of epinephrines, betablockers, direct miotics, cholinesterase inhibitors, carbonic anhydrase inhibitors, and prostoglandins and prostamides.

20. The punctal plug of claim 19 wherein the prostaglandins and prostamides are selected from one or more of latanoprost, bimatoprost, uravoprost, and unoprostone cidofovir.
